(19) United States
(12) Patent Application Publication
(10) Pub. No.: US 2020/0286622 A1
(43) Pub. Date: Sep. 10, 2020

(54) DATA ANALYSIS METHODS AND SYSTEMS FOR DIAGNOSIS AIDS

(71) Applicants: GACHON UNIVERSITY OF INDUSTRY-ACADEMIC COOPERATION FOUNDATION, Seongnam-si (KR); GIL MEDICAL CENTER, Incheon (KR)

(72) Inventors: Sungwon Jung, Incheon (KR); Sora Kim, Incheon (KR)

(73) Assignees: GACHON UNIVERSITY OF INDUSTRY-ACADEMIC COOPERATION FOUNDATION, Seongnam-si (KR); GIL MEDICAL CENTER, Incheon (KR)

(21) Appl. No.: 16/879,584

(22) Filed: May 20, 2020

Related U.S. Application Data
(63) Continuation-in-part of application No. PCT/KR2018/016983, filed on Dec. 31, 2018.

(30) Foreign Application Priority Data
Nov. 29, 2018 (KR) 10-2018-0150599

(51) Int. Cl. G16H 50/20; G16H 50/30; G16H 10/60; G16B 20/00; G16B 30/00; G06F 17/18

(52) U.S. Cl. CPC G16H 50/20; G16H 50/30; G06F 17/18; G16B 20/00; G16B 30/00; G16H 10/60

(57) ABSTRACT

The present invention relates to a data analysis method and system for disease diagnosis aid, and more specifically, to a technique and system capable of providing analysis results through integrated analysis of clinical, MRI images, and genotypic data to aid in disease diagnosis. The method includes receiving medical data of a subject; selecting disease-related data using the medical data; and calculating the disease probability according to the selected disease-related data. The medical data provides a configuration including clinical records, genetic and genetic variants, and MRI.

### CROSS-REFERENCE TO RELATED APPLICATIONS

This is a continuation-in-part of PCT/KR2018/016983, filed Dec. 31, 2018, which claims priority under 35 U.S.C. § 119 to Korean Patent Application No. 10-2018-0150599, filed on Nov. 29, 2018, which are both are hereby incorporated by reference in their entirely.

### TECHNICAL FIELD

The present invention relates to a data analysis method and system for disease diagnosis aid, and more particularly, to a technique and system capable of providing analysis results through integrated analysis of clinical, MRI images, and genomic data in order for disease diagnosis aid.

### BACKGROUND ART

Existing systems to aid in the diagnosis of a disease require phenotypic data and genotypic data, and analyze the data and provide a service that recommends the candidate disease name of the patient. Examples of systems that provide the service include Phenomizer, GenIO, and PhenoVar.

Phenomizer provides the function to show a candidate disease list with high correlation with the patient's phenotypic data by calculating the similarity between the patient's phenotypic data and the phenotypic data provided from the published disease database. However, since only a function for predicting a candidate disease list by using only the phenotypic data of the patient is provided in the case of the phenomizer, there is a disadvantage in that additional tools or systems are required to be used together with the actual patient's genetic data.

GenIO is a system developed to assist in the diagnosis process for rare genetic diseases, and provides services to find disease-causing variants of patients after analyzing clinical data and genotypic data. In order to provide the service, GenIO uses a program called Phenolyzer to obtain a candidate gene list associated with the inputted phenotypic data and find the variant that causes the patient's disease through filtering the genotypic data of the input patient based on the information and classification work according to mode of inheritance, pathogenicity, etc. However, in the case of the system, the size of the analysis and usable genotypic data is limited to 200 MB, and both the clinical and genotypic data are essential for data analysis. In addition, since a list of variants that cause a patient's disease is provided as a result of analysis, there is a disadvantage in that additional effort is required to find information on the variant in order to utilize it in actual diagnosis.

PhenoVar is also a system designed to achieve the goal of helping healthcare professionals to diagnose patients and the corresponding system provides a service that predicts a candidate disease of a real patient using clinical and genotypic data. PhenoVar uses an algorithm to quantify the association with specific diseases for each clinical and genotypic data to calculate the weight representing the association with a specific disease according to each data type, integrates the calculated weights, and provides a candidate disease list based on the final diagnostic score calculated for each disease. However, PhenoVar has several drawbacks. It is designed to input only the information belonging to several sub-categories provided by PhenoVar when inputting the patient's phenotypic data so that the phenotypic data available is limited. In addition, the local database used for clinical data analysis has a limitation that most of them are simulated patient's phenotypic data based on published disease related databases rather than actual patient data. In addition, like the GenIO system, the system has the disadvantage of requiring clinical and genotypic data.

As described above, most of the existing systems are developed based on analysis methods using clinical and genotypic data, and there are limitations in available input data formats or sizes. Furthermore, most existing systems require the input of specific data formats for analysis. Due to these problems, it is inconvenient for clinicians to use the system as an aid tool for patient diagnosis in a real clinical environment. For example, when presenting indirect evidence as a result, rather than direct evidence of a patient's candidate disease, or when considering additional types of data that are not supported by the existing system to use it for diagnosis, additional efforts and tools are needed to process the data. In addition, when there is no data required by the system, there is also a problem that the service provided by the system cannot be used.

Therefore, a system that provides services for aid in precise diagnosis of patients requires a system having no particular limitation on the input data format and including an integrated analysis method according to various input data.

### DISCLOSURE OF THE INVENTION

### Technical Problem

Accordingly, the present invention is to solve the above problems, and aims to develop and construct a system including an analysis method capable of integrating genomic, clinical, and MRI data for disease diagnosis aid.

### Technical Solution

A method for analyzing data for disease diagnosis aid according to an embodiment of the present invention for solving the above problems may include receiving, by a processor of a computer, medical data of a subject; selecting, by the processor, disease-related data using the medical data; and calculating, by the processor, a disease probability according to the selected disease-related data, wherein the medical data may include at least two or more of clinical records, genes and genetic variants, or MRI.

According to an embodiment of the present invention, the selecting of the disease-related data may include selecting a genome variant having a possibility of disease association among all genes and gene variants of the subject.

According to an embodiment of the present invention, the calculating of the disease probability may include: calculating a probability that the gene and gene variants selected by the processor are disease-related information; calculating an average rank of the selected genes according to the probability; and calculating a disease gene probability according to the number of disease candidate genes of the subject.

According to an embodiment of the present invention, the selecting of the disease-related data may include selecting a volume value of the MRI, a white matter damage volume value, a cortical and subcortical region T2 high signal damage volume value, and a myelination index, and the calculating of the probability may include: calculating the selected data and data of MRI of a previously stored disease-specific target case as a vector-based similarity percentile; and calculating an average value of the similarity percentiles.

According to an embodiment of the present invention, the calculating of the probability may include: evaluating a phenotype based similarity of the clinical information; and calculating a disease probability according to the similarity.

A data analysis system for disease diagnosis aid according to an embodiment of the present invention for solving the above problems may include: an input unit configured to receive medical data of a subject; a selection unit configured to select disease-related data using the medical data; and a disease detection unit configured to calculate a disease probability according to the selected disease-related data, wherein the medical data may include at least two or more of clinical records, genes and genetic variants, or MRI.

According to an embodiment of the present invention, the selection unit may select a genome variant having a possibility of disease association among all genes and gene variants of the subject.

According to an embodiment of the present invention, the calculating of the disease probability may include calculating a probability that the gene and gene variants selected by the processor are disease-related information; calculating an average rank of the selected genes according to the probability; and calculating a disease gene probability according to the number of disease candidate genes of the subject.

According to an embodiment of the present invention, the selection unit may select a volume value of the MRI, a white matter damage volume value, a cortical and subcortical region T2 high signal damage volume value, and a myelination index, and the disease detection unit may calculate the selected data and data of MRI of a previously stored disease-specific target case as a vector-based similarity percentile, and calculate an average value of the similarity percentiles.

According to an embodiment of the present invention, the disease detection unit may evaluate a phenotype based similarity of the clinical information, and calculate a disease probability according to the similarity.

### Advantageous Effects

According to the invention, it is possible to provide an integrated database that can utilize data from disease cohorts and published databases created through actual research and, based on this, obtain data that can be used when analyzing various types of patient data.

In addition, the present invention provides an analysis method including a method for quantitatively evaluating patient data of various types and capable of selectively combining and analyzing various types of patient data.

According to the above-described database and analysis method, a system usable in various clinical environments can be provided. In addition, the system provides a service that can shorten patient diagnosis time for clinicians based on various patient data.

Moreover, the effects of the present invention are not limited to the effects mentioned above, and various effects can be included within the scope of what will be apparent to a person skilled in the art from the following description.

### BRIEF DESCRIPTION OF THE DRAWINGS

FIG. 1 is a conceptual diagram of a data analysis system for disease diagnosis aid according to an embodiment of the present invention.

FIG. 2 is a block diagram of a data analysis system for disease diagnosis aid according to an embodiment of the present invention.

FIG. 3 is an example of calculating disease probability using genotypic data according to an embodiment of the present invention.

FIG. 4 is an example of calculating disease probability using clinical data according to an embodiment of the present invention.

FIG. 5 is an example of calculating disease probability using MRI data according to an embodiment of the present invention.

FIG. 6 is an example of the results in a phenotype-based similarity analysis according to an embodiment of the present invention.

FIG. 7 is an example of results in a phenotype-based similarity analysis according to an embodiment of the present invention.

FIG. 8 is an example of the results in a phenotype-based similarity analysis according to an embodiment of the present invention.

FIG. 9 is a flowchart of a data analysis method for disease diagnosis aid according to an embodiment of the present invention.

FIG. 10A shows analysis results using only genotypic data, FIG. 10B shows analysis results using only clinical record data, and FIG. 10C shows analysis results using genotypic data and clinical record data according to an embodiment of the present invention.

FIG. 11 shows an analysis result using a data analysis method and system for disease diagnosis aid according to an embodiment of the present invention.

### MODE FOR CARRYING OUT THE INVENTION

Hereinafter, a ‘data analysis method and system for disease diagnosis aid’ according to the present invention will be described in detail with reference to the accompanying drawings. The described embodiments are provided so that those skilled in the art can easily understand the technical spirit of the present invention, and the present invention is not limited thereby. In addition, matters expressed in the accompanying drawings may be different from shapes actually implemented as schematic drawings to easily describe embodiments of the present invention.

Moreover, each component represented below is only an example for implementing the present invention. Accordingly, other components may be used in other implementations of the present invention without departing from the spirit and scope of the present invention.

In addition, each component may be implemented solely in the configuration of hardware or software, but may also be implemented in a combination of various hardware and software components that perform the same function. Also, two or more components may be implemented together by one hardware or software.

In addition, the expression ‘includes’ certain components, as an expression of ‘open’, simply refers to the existence of the components, and should not be understood as excluding additional components.

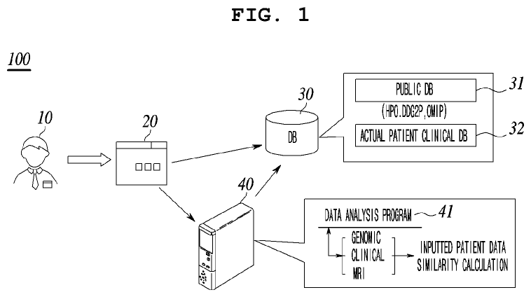

FIG. 1 is a conceptual diagram of a data analysis system for disease diagnosis aid according to an embodiment of the present invention.

Referring to FIG. 1, in relation to a data analysis system 100 for a disease diagnosis aid according to an embodiment of the present invention, a user 10 may aid in the disease diagnosis for actual patient data inputted using a database 30 and a data analysis program 41 through a user terminal 20.

The data analysis system 100 for disease diagnosis aid according to an embodiment of the present invention can solve the limitations of the database used in the analysis of several existing systems by using a separate database 30 created with reference to a public database related to diseases that provide clinical, genomic, MRI data and information related to developmental disorders of actual patients diagnosed with brain neurological developmental disorders.

By inventing independent analysis methods of genomic, clinical, and MRI data and methods of integrating and analyzing results from the process, it is possible to solve the limited input data format problem of existing systems.

The corresponding system was developed to provide services to perform an aid role in the accurate diagnosis process of patients who are expected to suffer from diseases of the brain nervous system development, and provides the function to search the candidate disease list of the patient by analyzing genomic, clinical, and MRI data for the corresponding service.

The system described above may include a data analysis program for performing the corresponding function as shown in FIG. 1, a self-curated database 30 for storing and managing data required for performing functions, and a program implemented by a self developed data analysis method.

The database 30 of the above-described system includes three types of data to store Evidence information of clinical and causal genes of diseases associated with diseases of the brain and nervous system development disorders necessary for performing a search function for a candidate disease of a patient. One can include data for storing data of Evidence created based on Human Phenotype Ontololgy (HPO), The Development Disorder Genotype-Phenotype Database (DDG2P), and Online Mendelian Inheritance in Man (OMIM), that is, the public database 31, and Evidence created based on clinical, genomic, and MRI data, which are the data of patients diagnosed with real brain nervous system development disorder.

HPO, used in Evidence information based on public databases, is a project that provides vocabulary for standardizing clinical data occurring from human disease, and as part of the corresponding project, provides standardized clinical data and a database containing information on diseases related to clinical data, and HPO included in the above-described database includes clinical and genetic information associated with OMIM-based cerebral nervous system development disorder diseases, including information on genetic diseases and clinical data stored in basic standardized terms. In addition, in order to quantitatively view differences in clinical data, several pieces of information for utilizing ontology-based similarity evaluation are added together and stored.

DDG2P is part of the Deciphering Developmental Disorders (DDD) project to analyze and study genomic and clinical data of children and parents with developmental disabilities in the UK and may provide standardized forms of clinical data in terms of disease-causing genes for developmental disorders and HPO terms observed in patients with actual diagnosis. The above-described database may include data such as clinical data, disease-causing genes, and mode of inheritance for brain neurological development diseases provided by DDG2P.

A wide range of information on the causative genes, genetic patterns, and patient symptoms reported so far for each hereditary disease based on previous reports of hereditary diseases is included in OMIM.

The above-described database may include clinical, genomic, and MRI data 32 of patients with actual brain neurological disease diagnosis. The actual patient's clinical data 32 may include the diagnosis name, disease cause gene, variant information, observed clinical abnormality of the patient in HPO terminology, and the like. The actual patient genotypic data contains variant information that causes the patient's disease, and actual patient MRI data may store information on brain structure features derived through data processing and analysis except for some very characteristic cases, due to the structure that is not accurate and detailed to describe in HPO

The above-described database may include a portion for storing evidence data for each inputable data and patient analysis results to search for a candidate disease of a patient based on an analysis result considering one or more input data.

The data analysis program of the above-described system may include a function of analyzing and storing a patient's data inputted by a clinician in an analytically usable form and combining and analyzing the results of each analyzed data.

The data analysis program 41 may be stored in the user terminal 20 or may be stored in the server 40. When the data analysis program 41 is stored in the server 40, data processing may be requested through communication.

Analysis methods using genomic and clinical data are applied to existing systems to aid patient's precise diagnosis. In addition, in order to use the corresponding analysis method, there are problems such as a limitation in which utilization data for each system is required essentially or is required essentially when inputting specific data. However, the data analysis program described above includes an analysis method that can additionally utilize MRI data in addition to the data used by the existing system, and a function to combine and analyze the analysis results, and the functions for processing and analyzing each data format are modularized. This analysis method and structure has a distinct advantage from the existing system. Unlike the existing system, the data analysis program having the analysis method and structure described above allows medical workers to directly select data available for patient diagnosis, and by providing a data processing and analysis method according to the selected data, the system described above can provide a service that can be used in various clinical environments.

The data analysis program 41 according to an embodiment of the present invention may calculate disease similarity to actual patient data inputted to the system by using the genomic DB, clinical DB, and MRI DB stored in the public database 31 and the actual patient clinical database 32

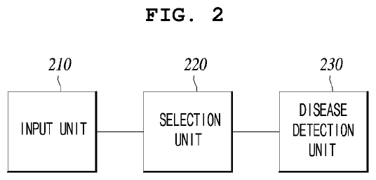

FIG. 2 is a block diagram of a data analysis system for disease diagnosis aid according to an embodiment of the present invention.

Referring to FIG. 2, a data analysis system for disease diagnosis aid according to an embodiment of the present invention may include an input unit 210, a selection unit 220 and a disease detection unit 230.

The input unit 210 may receive medical data of an examinee. The medical data received by the input unit 210 may include clinical records, genes and genetic variants, and MRI. The data may be inputted in a computer-readable form. The input unit 210 may preprocess the medical data in a form that can be processed by the selection unit 220 or the disease detection unit 230 and transfer the preprocessed medical data.

The selection unit 220 may receive the medical data from the input unit 210. The selection unit 220 may select disease-related data using the medical data. Information included in the medical data may be selected.

The selection unit 220 may select a variant having a possibility of disease association among all gene variants possessed by the subject. The selection unit 220 may select the subject's brain region volume value, white matter damage volume value, cortex and subcortical region T2 high signal damage volume value, and myelination index from MRI data.

The disease detection unit 230 may calculate the disease probability according to the selected disease-related data. The disease detection unit 230 may provide an expected disease according to the probability of the disease. The disease detection unit 230 may calculate a disease probability according to a plurality of types of the disease-related data, and may determine a disease probability or a predicted disease in consideration of the calculated multiple disease probability.

The disease detection unit 230 synthesizes the results of pathogenicity prediction tools to determine the probability that the variant vⱼ is a pathogenic variant with respect to the selected genome variants and calculates it as P(vⱼ=pathogenic variant|prediction result of pathogenicity of vⱼ).

If the gene gᵢ variant has multiple vⱼ, the disease detection unit 230 may obtain the disease gene probability of this gene gᵢ as the maximum value of the pathogenic variant probability of each variant as follows. P(gᵢ=disease gene)=max(P(vⱼ=pathogenic variant|pathogenicity prediction result of vⱼ))

The disease detection unit 230 may obtain the average rank rᵢ of the disease gene probability P(gᵢ=disease gene) of each gene gᵢ, for the disease candidate genes possessed by the subject.

If the disease candidate genes possessed by the subject are N, the disease detection unit 230 may calculate the normalized disease gene probability P_{N}(gᵢ=disease gene) of the gene gᵢ as shown in Equation 1 below.

1−(rᵢ−1)/max(rᵢ) [Equation 1]

If the disease gene specified in the Evidence is gk, since it is clear that this Evidence disease gene is gk, the disease detection unit 230 may assume that the normalized disease gene probability is 1. At this time, the genetic variant based similarity between the patient and this Evidence can be determined as min(P_{N}(gk=disease gene), 1). 1) However, this is the case where the patient's allelic status and genetic pattern of the gene gk variant are consistent with those specified in Evidence, otherwise the similarity can be determined as 0. If the comparison with the subject is another patient B whose disease gene has not been identified, the genetic variant similarity in terms of gk between the two patients can be determined as follows. min(P_{N}(gk=disease gene), P_{N}^{B}(gk=disease gene)).

The disease detection unit 230 may satisfy all of the following criteria for variants that may be associated with disease. 1) located in the exonic or splicing region, 2) should not be a synonymous variant, 3) the frequency of detection is less than 0.5% in all known population cohorts. It should be listed as a disease-causing gene in OMIM, and the allelic status of the variant should be consistent with the genetic pattern of the corresponding disease.

In order to calculate the pathogenic probability of each variant, the disease detection unit 230 may utilize the pathogenicity information of the previously reported disease gene variant DB, ClinVar, and prediction information of the following pathogenicity prediction tools: SIFT, Polyphen2, LRT, MutationTaster, MutationAssessor, FATHMM, RadialSVM, LR.

In the disease detection unit 230, the probability P(vⱼ=pathogenic variant|pathogenicity prediction result of vⱼ) that a variant vⱼ is a pathogenic variant can be obtained by averaging Pₜ(vⱼ=pathogenicity prediction result of vⱼ by pathogenic variant|t) obtained by each prediction tool t. At this time, Pₜ(vⱼ=pathogenicity prediction result of vⱼ by pathogenic variant|t) can be calculated as follows by Bayes' theorem. Pₜ(vⱼ=pathogenic variant|pathogenicity prediction result of vⱼ by t)=Pₜ(pathogenicity prediction result of vⱼ by t|vⱼ=pathogenic variant)×P(vⱼ=pathogenic variant)/P(pathogenicity prediction result of vⱼ by t)

Pₜ(pathogenicity prediction result of vⱼ by t|vⱼ=pathogenic variant) for use in the above calculation can be calculated by assuming that the older version of the two versions of ClinVar having different differences is prediction and the latest version is actual variant information. This calculation can be done by learning the naive Bayes classifier by using each gene variant described in ClinVar as one input data and using the prediction information of pathogenicity prediction tools for the corresponding gene variant as a parameter constituting the corresponding input data.

P(vⱼ=pathogenic variant) and P(pathogenicity prediction result of vⱼ by t) can be estimated from 69,499,850 gene variants present in a total of 127 patient whole exome-sequencing data.

The disease detection unit 230 may calculate the similarity through the phenotype-based similarity evaluation of the clinical information. The disease detection unit 230 may calculate a disease probability using the similarity. The disease detection unit 230 may present the predicted disease using the similarity or disease probability.

The disease detection unit 230 may calculate the similarity through a total of 35 phenotype term list-to-term list similarity calculation techniques according to a combination of seven phenotype term-to-term similarity evaluation techniques secured by software libraries, such as Resnick, Lin, Jiang-Conrath, relevance, information coefficient, graph IC, and Wang, and five similarity combining techniques that can be used for term set-to-term set similarity calculation, such as Max, Mean, funSim Max, FunSimAvg, and BMA.

In order to find the best of 35 similarity evaluation techniques, based on 151 patients' disease information and phenotype, the disease detection unit 230 may evaluate the ranking of the same disease by calculating phenotype similarity for other cases of each case through a leave-one-out cross-validation method.

The disease detection unit 230 may calculate a percentile of the vector-based similarity of each of the disease-related data classifications selected from MRI data of the subject and MRI data of comparison cases, and may obtain the average value of the percentile similarity calculated for each classification.

The disease detection unit 230 may obtain an average rank rᵢ between the input case and the comparison target data based on the calculated average value of the similarity percentile, and based on this, may finally calculate the normalized similarity value 1−(rᵢ−1)/max(rᵢ).

The disease detection unit 230 may calculate normalized similarity values of input patient data and reference data (e.g., SNU cohort or DDD project data) in the platform for each data type through the above processes.

When all or a part of the similarity for each data type is selected and combined, the disease detection unit 230 may calculate a general similarity as an average of corresponding normalized similarity values.

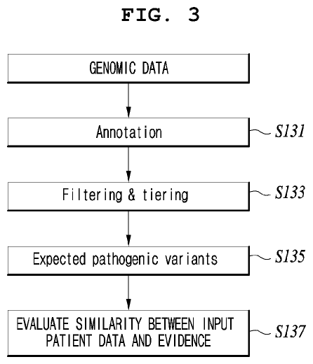

FIG. 3 is an example of calculating disease probability using genotypic data according to an embodiment of the present invention.

Referring to FIG. 3, genotypic data analysis of a patient may be performed through a system analysis process. Like most existing systems, the data analysis program described above may use a Variant Call Format (VCF) file, which is a standard file format used to store a genome variant, as an input.

The above-described program may perform an annotation operation (S131) for adding information on a variant using an input VCF file, and at this time, generate a result file in text format separated by tabs including information on the gene of the variant, frequency of the population level, the region of the variant, and pathogenic scores using the ANNOVAR program. Thereafter, additional information annotation and filtering operations may be performed using the result file generated by the annotation process (S133). The Filtering & Tiering process described above may use OMIM, a database of disease genes not supported by the ANNOVAR program, various logical expressions developed in-house to process genotype of variant, and Germline Variant Annotation Filtering (GVAF) software that provides annotation function based on text file rather than VCF format and genetic variant filtering through a combination of logical expressions, and additionally annotate disease information based on the genetic information of the variant using the corresponding software. In order to find disease-causing variants it is possible to filter and extract the variants that satisfy the conditions of the variants present in the exon or splicing region, which are observed with frequencies less than 0.05% of the database, in various Population levels.

Variants extracted by the filtering process can be classified according to the classification conditions of whether the variant is a direct disease cause or whether it is a variant of an existing disease-causing gene (S135).

Expected pathogenic variant process (S135) finds a variant that can cause disease after calculating the pathogenic score of the variant selected by the Filtering & Tiering process. Based on various variant information, including information generated by the process of expected pathogenic variants, by calculating the similarity with the Evidence stored in the database described above, quantitative evaluation of genotypic data between the input patient and Evidence can be performed (S137).

The process of quantitatively evaluating the similarity between the input patient data and the Evidence (S137) may calculate the similarity by comparing the Evidence information stored in the database with the genomic variant that causes the predicted disease.

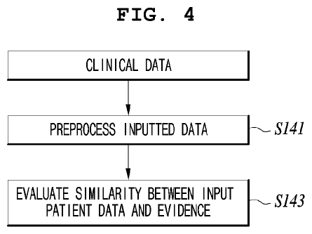

FIG. 4 is an example of calculating disease probability using clinical data according to an embodiment of the present invention.

Referring to FIG. 4, in the data analysis program described above, clinical data may be used to analyze the patient's clinical data through the system analysis process, and at this time, the input of clinical data may be performed using HPO Term name belonging to HPO, which is a standardized clinical term system.

The above-described program may analyze phenotypic data using an ontology-based similarity evaluation method, and obtain a term-term similarity by using information on the relationship between terms in the corresponding similarity evaluation method. For this, a preprocessing process for analyzing the input phenotypic data is performed (S141). The preprocessing process (S141) changes the data type for quantitative evaluation of actual phenotypic data, and the corresponding process is to change the data inputted in the form of HPO Term name into the form of HPO Term ID. For example, when the inputted phenotypic data is “Focal seizures, Global developmental delay, Intellectual disability”, it is changed to the corresponding HPO Term ID “0007359, 0001263, 0001249” corresponding to the corresponding HPO Term name that is converted through the preprocessing process.

The phenotypic data changed to the HPO Term ID is used as a self-developed program to calculate the similarity to the phenotypic data of Evidence stored in the above-described database, thereby performing quantitative evaluation of phenotypic data between the input patient and the Evidence (S143).

The similarity evaluation process (S143) between the input patient data and the Evidence data may calculate the similarity between the preprocessed clinical data of the patient and the Evidence data stored in the database.

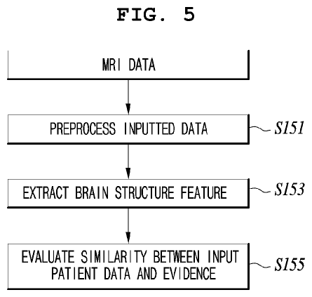

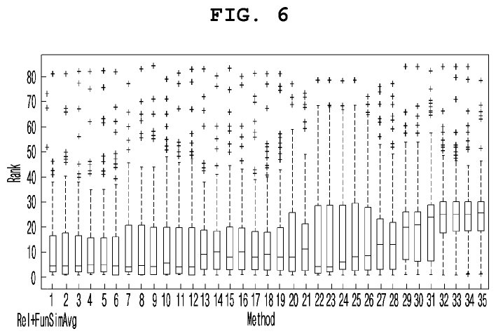

FIG. 5 is an example of calculating disease probability using MRI data according to an embodiment of the present invention.

Referring to FIG. 5, a program for processing and analyzing MRI data in the system may perform analysis using a method of quantitatively evaluating the similarity between the patient's MRI data and the Evidence stored in the database. For this, a preprocessing process for analyzing the input MRI data is performed (S151). In general, in relation to MRI data, due to various constraints and necessities of the clinical field, it is highly possible that a relatively low resolution 2D image is obtained instead of such a high resolution image, and these 2D images have a limitation that 2D images do not have much information that can be obtained when deriving the structural properties of the actual brain. In order to solve this, the preprocessing process may perform a preprocessing process of converting an existing 2D image into a high-resolution 3D image.

The image data obtained by the preprocessing process (S151) is used to derive direct attribute values related to brain neurological diseases and brain functional damage (S153).

By using software to extract features of the brain structure, data such as the volume of normal gray matter and white matter, the volume of the damaged white matter lesion, cortical thickness, cortical area, and curvature are derived (S153).

The derived attribute values are used to calculate the similarity of comparing with MRI data of actual patients stored in the above-described database. By calculating the similarity between the attribute value for the brain structure characteristic of the input patient and the Evidence data, quantitative evaluation of MRI data between the input patient and Evidence is performed (S155).

The analysis method includes a method of combining results evaluated by a data analysis process, and various patient data can be selectively used by utilizing the corresponding analysis method.

FIG. 6 is an example of the results in a phenotype-based similarity analysis according to an embodiment of the present invention.

FIG. 6 is an accuracy evaluation result of 35 phenotype similarity evaluation methods by leave-one-out cross-validation based on information of 151 patients. The 35 methods evaluated in FIG. 6 can confirm the distribution of the same disease ranking in 151 cases. In the case of the combination of the relevance method and the FunSimAvg similarity combining technique, it can be seen that it shows the highest ranking average. When comparing input patients with the cohort of Seoul National University Hospital on the platform developed based on this, it can be determined to evaluate the phenotype similarity by combining the Relevance method and FunSimAvg technique.

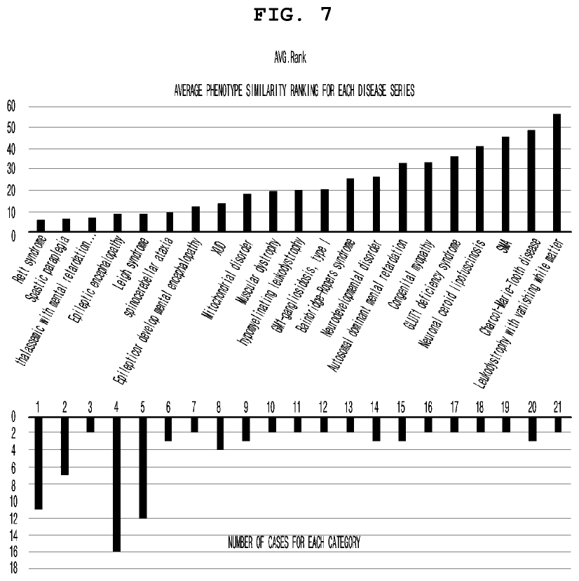

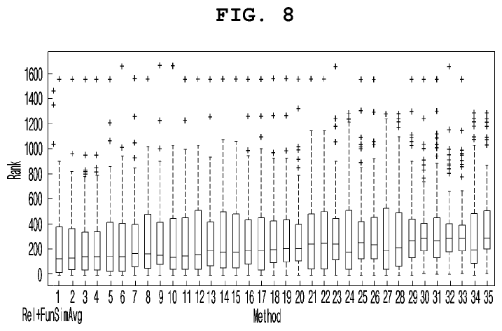

FIG. 7 is an example of the results in a phenotype-based similarity analysis according to an embodiment of the present invention.

Referring to FIG. 7, it is possible to confirm the tendency of the phenotype similarity evaluation according to the disease series-specific comparison target case number. FIG. 7 shows the number of patient data that existed in the disease for each series by obtaining the average by classifying the similarity ranking of the same disease for each disease series when combining the relevance method and the FunSimAvg similarity combining technique. Higher ranking may be shown in Rett syndrome, spastic paraplegia, epileptic encephalopathy, and Leigh syndrome, where relatively many patient cases are secured. Through this, it can be seen that securing more patient cases as reference data helps improve disease prediction performance.

FIG. 8 is an example of the results in a phenotype-based similarity analysis according to an embodiment of the present invention.

Referring to FIG. 8, 35 phenotype similarity techniques are evaluated by comparing phenotypes of 151 patients with phenotypes for each disease reported in the Deciphering Developmental Disorders (DDD) project.

FIG. 8 shows the distribution of rankings that each of the 35 phenotype similarity evaluation techniques evaluate for the same disease when comparing the 151 phenotypes with the phenotype information for each disease reported in the DDD project. In comparison with the phenotype information reported by the DDD project, the use of Resnick technique other than the relevance measure, which was excellent in leave-one-out cross-validation, was evaluated to be superior among 151 cases. In the case of phenotype information accompanying each of the 151 patient data, only the phenotype seen by each patient is recorded, but the phenotype for each disease reported in the DDD project is different because it records the phenotype reported for each disease so that differences may occur in suitable evaluation methods. Based on the above results, when searching the DDD project data for the phenotype of the input patient in the developed platform as a reference, a combination of the Resnick method and the FunSimAvg method can be employed as a phenotype similarity evaluation technique. Based on the similarity calculated by each method, the average rank rᵢ between the input case and the data to be compared is calculated, and based on this, the normalized similarity value 1−(r−1)/max(rᵢ) can be finally calculated. A data analysis method and system for disease diagnosis aid according to an embodiment of the present invention was confirmed to have a superior effect with an accuracy of 95.6% when Exomiser and PhenoVar, that is, the existing technologies, have an accuracy of 56% and 89%, respectively.

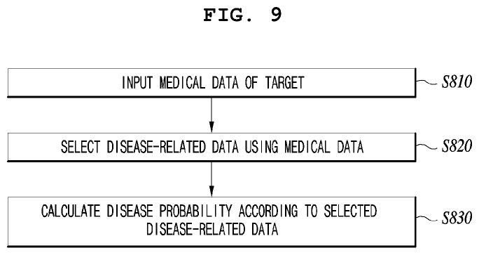

FIG. 9 is a flowchart of a data analysis method for disease diagnosis aid according to an embodiment of the present invention.

Referring to FIG. 9, a data analysis method for disease diagnosis aid according to an embodiment of the present invention may include receiving medical data of a subject (S910).

In operation S910, the input unit 210 may receive medical data of the examinee. The medical data received by the input unit 210 may include clinical records, genes and genetic variants, and MRI. The data may be inputted in a computer-readable form. The input unit 210 may preprocess the medical data in a form that can be processed by the selection unit 220 or the disease detection unit 230 and transfer the preprocessed medical data.

A data analysis method for disease diagnosis aid according to an embodiment of the present invention may include selecting the disease-related data using the medical data (S920).

In operation S920, the selection unit 220 may receive the medical data from the input unit 210. The selection unit 220 may select disease-related data using the medical data. Information included in the medical data may be selected.

The selection unit 220 may select a variant having a possibility of disease association among all gene variants possessed by the subject. The selection unit 220 may select the subject's brain region volume value, white matter damage volume value, cortex and subcortical region T2 high signal damage volume value, and myelination index from MRI data.

A data analysis method for disease diagnosis aid according to an embodiment of the present invention may include calculating the disease probability according to the selected disease-related data (S930).

In operation S930, the disease detection unit 230 may calculate the disease probability according to the selected disease-related data. The disease detection unit 230 may provide an expected disease according to the probability of the disease. The disease detection unit 230 may calculate a disease probability according to a plurality of types of the disease-related data, and may determine a disease probability or a predicted disease in consideration of the calculated multiple disease probability.

The disease detection unit 230 synthesizes the results of pathogenicity prediction tools to determine the probability that the variant vⱼ is a pathogenic variant with respect to the selected genome variants and calculates it as P(vⱼ=pathogenic variant|prediction result of pathogenicity of vⱼ).

If the gene gᵢ variant has multiple vⱼ, the disease detection unit 230 may obtain the disease gene probability of this gene gᵢ as the maximum value of the pathogenic variant probability of each variant as follows. P(gᵢ=disease gene)=max(P(vⱼ=pathogenic variant|pathogenicity prediction result of vⱼ))

The disease detection unit 230 may obtain the average rank rᵢ of the disease gene probability P(gᵢ=disease gene) of each gene gi, for the disease candidate genes possessed by the subject.

If the disease candidate genes possessed by the subject are N, the disease detection unit 230 may calculate the normalized disease gene probability P_{N}(gᵢ=disease gene) of the gene gᵢ as shown in Equation 1 below.

1−(rᵢ−1)/max(rᵢ) [Equation 1]

If the disease gene specified in the Evidence is gₖ, since it is clear that this Evidence disease gene is gₖ, the disease detection unit 230 may assume that the normalized disease gene probability is 1. At this time, the genetic variant based similarity between the patient and this Evidence can be determined as min(P_{N}(gₖ=disease gene), 1). 1) However, this is the case where the patient's allelic status and genetic pattern of the gene gₖ variant are consistent with those specified in Evidence, otherwise the similarity can be determined as 0. If the comparison with the subject is another patient B whose disease gene has not been identified, the genetic variant similarity in terms of gₖ between the two patients can be determined as follows. min(P_{N}(gₖ=disease gene), P_{NB}(gₖ=disease gene)).

The disease detection unit 230 may satisfy all of the following criteria for variants that may be associated with disease. 1) located in the exonic or splicing region, 2) should not be a synonymous variant, 3) the frequency of detection is less than 0.5% in all known population cohorts. It should be listed as a disease-causing gene in OMIM, and the allelic status of the variant should be consistent with the genetic pattern of the corresponding disease.

In order to calculate the pathogenic probability of each variant, the disease detection unit 230 may utilize linVar pathogenicity information and prediction information of the following pathogenicity prediction tools. SIFT, Polyphen2, LRT, MutationTaster, MutationAssessor, FATHMM, RadialSVM, LR

In the disease detection unit 230, the probability P(vⱼ=pathogenic variant|pathogenicity prediction result of vⱼ) that a variant vⱼ is a pathogenic variant can be obtained by averaging Pₜ(vⱼ=pathogenicity prediction result of vⱼ by pathogenic variant|t) obtained by each prediction tool t. At this time, Pₜ(vⱼ=pathogenicity prediction result of vⱼ by pathogenic variant|t) can be calculated as follows by Bayes' theorem. Pₜ(vⱼ=pathogenic variant|pathogenicity prediction result of vⱼ by t)=Pₜ(pathogenicity prediction result of vⱼ by t|vⱼ=pathogenic variant)×P(vⱼ=pathogenic variant)/P(pathogenicity prediction result of vⱼ by t)

Pₜ(pathogenicity prediction result of vⱼ by t|vⱼ=pathogenic variant) for use in the above calculation can be calculated by assuming that the older version of the two versions of ClinVar having different differences is prediction and the latest version is actual variant information.

P(vⱼ=pathogenic variant) and P(pathogenicity prediction result of vⱼ by t) can be estimated from 69,499,850 gene variants present in a total of 127 patient whole exome-sequencing data.

The disease detection unit 230 may calculate the similarity through the phenotype-based similarity evaluation of the clinical information. The disease detection unit 230 may calculate a disease probability using the similarity. The disease detection unit 230 may present the predicted disease using the similarity or disease probability.

The disease detection unit 230 may calculate the similarity through a total of 35 phenotype term list-to-term list similarity calculation techniques according to a combination of seven phenotype term-to-term similarity evaluation techniques secured by software libraries, such as Resnick, Lin, Jiang-Conrath, relevance, information coefficient, graph IC, and Wang, and five similarity combining techniques that can be used for term set-to-term set similarity calculation, such as Max, Mean, funSim Max, FunSimAvg, and BMA.

In order to find the best of 35 similarity evaluation techniques, based on 151 patients' disease information and phenotype, the disease detection unit 230 may evaluate the ranking of the same disease by calculating phenotype similarity for other cases of each case through a leave-one-out cross-validation method.

The disease detection unit 230 may calculate a percentile of the vector-based similarity of each of the disease-related data classifications selected from MRI data of the subject and MRI data of comparison cases, and may obtain the average value of the percentile similarity calculated for each classification.

The disease detection unit 230 may obtain an average rank ri between the input case and the comparison target data based on the calculated average value of the similarity percentile, and based on this, may finally calculate the normalized similarity value 1−(rᵢ−1)/max(rᵢ).

The disease detection unit 230 may calculate normalized similarity values of input patient data and reference data (e.g., SNU cohort or DDD project data) in the platform for each data type through the above processes.

When all or a part of the similarity for each data type is selected and combined, the disease detection unit 230 may calculate a general similarity as an average of corresponding normalized similarity values.

A data analysis method and system for disease diagnosis aid according to another embodiment of the present invention applies a weight to each evaluation value for clinical record data, genotypic data, and MRI data to diagnose the corresponding patient with a disease with the highest probability. Meanwhile, the following equation can be used as a method of applying the weight.

$$Pr ( D \vert P ) = {w}_{0} \times ecdf ( D : {max}_{i , j} \frac{{\sum }_{t = 1}^{T} {θ}_{t} P ( {v}_{ij} is pathogenic \vert {PathoPred}_{ t} )}{T} ) + {w}_{1} \times ecdf ( D : \frac{1}{2} [ \frac{1}{m} {\sum }_{i = 1}^{m} {max}_{3 \leq j \leq n} \{ Resnick ( {phenotype}_{pi} , {phenotype}_{Dj} ) \times min ( {freq}_{D} ( {phenotype}_{pi} ) , {freq}_{D} ( {phenotype}_{Dj} ) ) \} + \frac{1}{n} {\sum }_{j = 1}^{n} {max}_{3 \leq j \leq n} \{ Resnick ( {phenotype}_{pi} , {phenotype}_{Dj} ) \times min ( {freq}_{D} ( {phenotype}_{pi} ) , {freq}_{D} ( {phenotype}_{Dj} ) ) \} ] ) + {w}_{2} \times ecdf ( {D}_{i} \sqrt{{\sum }_{I} {{γ}_{f} ( {MRI}_{f}^{F} - {MRI}_{f}^{D} )}^{2}} )$$

Here, ecdf(x; z) is defined as an empirical cumulative distribution function for z, P means an input patient, D means a type of disease, Pr( ) means probability, w0 is a weight for genotypic data, w1 is a weight for phenotype information, w2 is a weight for MRI data, and w0+w1+w2=1 is satisfied, and each variable is defined as follows.

T: The number of prediction tools PathoPred that predict the pathogenicity of genetic variants,

θₜ: Weight for the t-th prediction tool PathoPredt. Σθₜ=1

vᵢⱼ: j-th variant of patient P for the i-th gene reported to induce disease D

m: The number of phenotypes observed in patient P

n: The number of phenotypes reported in disease D

phenotype_{Pi}: i-th phenotype of patient P

phenotype_{Dj}: j-th phenotype reported in disease D

freq_{D}(phenotype): Frequency of phenotypes reported in disease D

MRI_{f}^{P}: f-th feature of patient P MRI data vector

MRI_{f}^{D}: f-th feature of disease D MRI data vector

γ_{f}: Weight for f-th feature of MRI data vector. Σγ_{f}=1

P(vᵢⱼ is pathogenic|PathoPredₜ) represents the disease-induced probability of gene variant by each pathogenicity prediction tool, and it is possible that this probability value is estimated from previously reported pathogenic gene variant information DB and normal human gene variant information.

The values of the weights w0, w1, and w2 can be set as follows, and the weights can be adjusted according to the purpose.
w0=1, w1=0, w2=0: When only genotypic data is used
w0=0, w1=1, w2=0: When only Phenotype information is used
w0=0.5, w1=0.5, w2=0: When genotypic data and phenotype data are used
w0=⅓, w1=⅓, w2=⅓: When all data is used

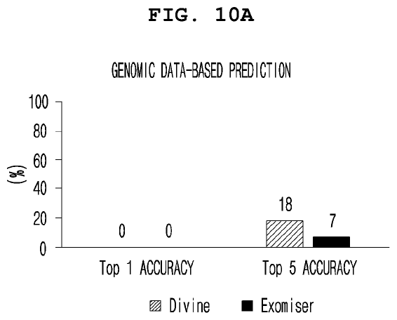

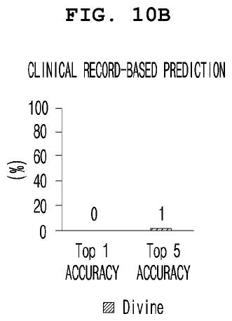

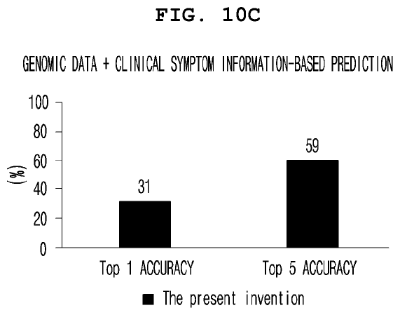

FIG. 10A shows analysis results using only genotypic data, FIG. 10B shows analysis results using only clinical record data, and FIG. 10C shows analysis results using genotypic data and clinical record data according to an embodiment of the present invention.

More specifically, referring to FIG. 10A shows the results of analysis with Divine and Exomiser, that is, conventional analysis programs, using only genotypic data, and FIG. 10B shows the results of analysis with Divine, that is, a conventional analysis program, using only clinical record data.

Top1 means the accuracy of the disease corresponding to the 1st rank among the predicted diseases, and Top5 means the accuracy of being the actual disease among diseases corresponding to 5th among the predicted diseases.

According to FIG. 10A, the result of the Divine program using only genomic data showed accuracy of 0% in Top1 (left bar of Top1) and 18% in Top5 (left bar of Top 5), and the results of the Exomiser program showed accuracy of 0% in Top1 (right bar of Top1) and 7% in Top5 (right bar of Top 5). According to FIG. 10B, the results of the Divine program using only clinical symptom information showed accuracy of 0% and 1% in Top1 and Top5, respectively. For reference, the conventional Exomiser program cannot derive prediction results using only clinical symptom information.

Referring to FIG. 10C, the results of using genotypic data and clinical symptom information together with a data analysis method/system according to an embodiment of the present invention showed an accuracy of 31% in Top1 and 59% in Top5.

Compared to FIGS. 10A and 10B, it can be seen that the prediction result according to the present invention is much higher than results using only genomic data (0% for Top1, 18% for Top5) and results using only clinical symptom information (0% for Top1, 1% for Top5). Even if using genotypic data and clinical symptom information together to predict through a conventional analysis program, while the prediction result of the conventional analysis program was 19.78%, since prediction accuracy of 31% to 33% in Top 1 and 59% to 62% in Top 5 can be derived by a data analysis method according to an embodiment of the present invention, it was confirmed that the data analysis method according to the present invention is superior to the conventional method. Therefore, according to an embodiment of the present invention, when two or more types of data, such as genotypic data and clinical record data, are used together, disease diagnosis prediction performance can be improved.

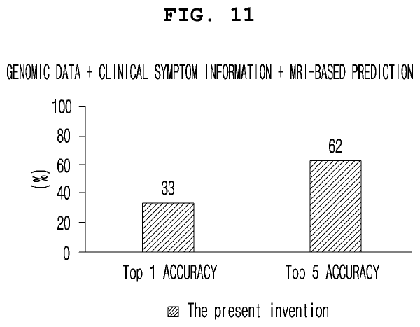

FIG. 11 shows an analysis result using a data analysis method and system for disease diagnosis aid according to an embodiment of the present invention.

More specifically, referring to FIG. 11, disease genotypic data, clinical record data, and MRI data are all used to represent disease diagnosis prediction results according to an embodiment of the present invention, and the accuracy is 33% in Top1 and 62% in Top5. Therefore, it can be seen that the prediction performance of disease diagnosis can be improved than the prediction performance of the conventional programs shown in FIGS. 10A and 10B.

So far, the present invention has been focused on the preferred embodiments. Those skilled in the art to which the present invention pertains will appreciate that the present invention may be implemented in a modified form without departing from the essential characteristics of the present invention. Therefore, the disclosed embodiments should be considered in descriptive sense only not in limited perspective sense. The scope of the invention is defined not by the detailed description of the invention but by the appended claims, and all differences within the scope will be construed as being included in the present invention.

1. A data analysis method for aiding disease diagnosis, comprising:
receiving, by a processor of a computer, medical data of a subject;
selecting, by the processor, disease-related data using the medical data; and
calculating, by the processor, a disease probability according to the selected disease-related data,
wherein the medical data comprises at least two or more of a) a clinical record, b) genes and genetic variants, or c) MRI data, and
wherein a) when the medical data is the clinical record, the calculating of the disease probability comprises:
evaluating, by the processor, a phenotype-based similarity of the clinical information; and
calculating, by the processor, the disease probability according to the phenotype-based similarity,
wherein b) when the medical data is the genes and gene variants, the selecting of the disease-related data comprises selecting a genome variant having a possibility of disease association among all genes and gene variants of the subject, and the calculating of the disease probability comprises:
calculating, by the processor, a probability that the genes and gene variants selected by the processor are disease-related information;
calculating, by the processor, an average rank ri_1 of the selected genes according to the probability;
calculating, by the processor, a disease gene probability P_1 according to a number of disease candidate genes of the subject; and
calculating a normalized probability (1−(ri_1−1)/max (ri_1)) of the disease gene probability P_1,
wherein c) when the medical data is the MRI data, the selecting of the disease-related data comprises selecting, by the processor, a volume value of the MRI, a white matter damage volume value, a cortical and subcortical region T2 high signal damage volume value, and a myelination index as selected data, and the calculating of the probability comprises:
calculating, by the processor, the selected data and data of MRI of a previously stored disease-specific target case as a vector-based similarity percentile;
calculating, by the processor, an average value of the vector-based similarity percentiles; and
calculating, by the processor, a similarity average rank ri_2 and a normalized similarity value 1−(ri_2−1)/max (ri_2) between an input case and comparison target data based on the average value of the similarity percentiles.

2. The method of claim 1, wherein the calculating, by the processor, the disease probability comprises applying a weight to each evaluation value for clinical record data, genes and gene variants, and MRI data,
wherein the applying of the weight is performed using the following equation:
$$Pr ( D \vert P ) = {w}_{0} \times ecdf ( D \text{:} {max}_{i , j} \frac{{\sum }_{\text{?} = 1}^{T} {θ}_{t} P ( {v}_{\text{?}} is pathogenic \vert {Path}_{\text{?}} {Pred}_{t} )}{T} ) + {w}_{\text{?}} \times ecdf ( D \text{:} \frac{1}{2} [ \frac{1}{m} {\sum }_{i = 1}^{\text{?}} {max}_{1 \leq j \leq n} \{ Resnick ( {phenotype}_{\text{?}} , {phenotype}_{\text{?}} ) \times min ( {freq}_{\text{?}} ( {phenotype}_{\text{?}} ) \text{,} {freq}_{\text{?}} ( {phenotype}_{\text{?}} ) ) \} + \frac{1}{n} {\sum }_{j = 1}^{\text{?}} {max}_{1 \leq \text{?} \leq \text{?}} \{ Resnick ( {phenotype}_{\text{?}} , {phenotype}_{\text{?}} ) \times min ( {freq}_{\text{?}} ( {phenotype}_{\text{?}} ) , {freq}_{\text{?}} ( {phenotype}_{} ) ) \} ] ) + {w}_{2} \times ecdf ( D \text{:} \sqrt{{\sum }_{f} {{γ}_{f} ( {MRI}_{\text{?}}^{\text{?}} - {MRI}_{f}^{} )}^{2}} ) \\ \text{?} \text{indicates text missing or illegible when filed}$$
(where ecdf(x; z) is an empirical cumulative distribution function for z, P is an input patient, D is a type of disease, Pr( ) is probability, w0 is a weight for genes and gene variants, w1 is a weight for phenotype information, w2 is a weight for MRI data, and w0+w1+w2=1 is satisfied, and each variable is defined as follows:
T: The number of prediction tools PathoPred that predict the pathogenicity of genetic variants,
θₜ: Weight for the t-th prediction tool PathoPredt Σθₜ−1
vᵢⱼ: j-th variant of patient P for the i-th gene reported to induce disease D
m: The number of phenotypes observed in patient P
n: The number of phenotypes reported in disease D
phenotype_{Pi}: i-th phenotype of patient P
phenotype_{Dj}: j-th phenotype reported in disease D
freq_{D}(phenotype): Frequency of phenotypes reported in disease D
MRI_{f}^{P}: f-th feature of patient P MRI data vector
MRI_{f}^{D}: f-th feature of disease D MRI data vector
γ_{f}: Weight for f-th feature of MRI data vector Σγ_{f}=1).

3. A data analysis system for aiding disease diagnosis, comprising:
an input unit that receives medical data of a subject;
a selection unit that selects disease-related data using the medical data; and
a disease detection unit that calculates a disease probability according to the selected disease-related data,
wherein the medical data comprises at least two or more of a) clinical records, b) genes and genetic variants, or c) MRI data,
wherein a) when the medical data is a clinical record, the disease detection unit evaluates a phenotype based on similarity of the clinical information, and calculates a disease probability according to the similarity,
wherein b) when the medical data is the genes and gene variants, the selection unit selects a gene or a gene variant having a possibility of disease association among all genes and gene variants of the subject,
and the disease detection unit calculates a probability that the selected gene or gene variant are disease-related information, calculates an average rank ri_1 of the selected gene according to the probability, calculates a disease gene probability P_1 according to a number of disease candidate genes of the subject, and calculates a normalized probability (1−(ri_1−1)/max (ri_1)) of the disease gene probability P_1,
wherein c) when the medical data is MRI data, the selection unit selects a volume value of the MRI, a white matter damage volume value, a cortical and subcortical region T2 high signal damage volume value, and a myelination index as selected data,
and the disease detection unit calculates the selected data and data of MRI of a previously stored disease-specific target case as a vector-based similarity percentile, calculates an average value of the similarity percentiles, and calculates a similarity average rank ri_2 and a normalized similarity value 1−(ri_2−1)/max (ri_2) between an input case and comparison target data based on the average value of the similarity percentiles.

4. The system of claim 3, wherein the calculating, by the disease detection unit, of the disease probability comprises applying a weight to each evaluation value for clinical record data, genes and gene variants, and MRI data,
wherein the applying of the weight follows the following equation:
$$Pr ( D \vert P ) = {w}_{0} \times ecdf ( D \text{:} {max}_{i , j} \frac{{\sum }_{\text{?} = 1}^{T} {θ}_{t} P ( {v}_{\text{?}} is pathogenic \vert {Path}_{\text{?}} {Pred}_{t} )}{T} ) + {w}_{\text{?}} \times ecdf ( D \text{:} \frac{1}{2} [ \frac{1}{m} {\sum }_{i = 1}^{\text{?}} {max}_{1 \leq j \leq n} \{ Resnick ( {phenotype}_{\text{?}} , {phenotype}_{\text{?}} ) \times min ( {freq}_{\text{?}} ( {phenotype}_{\text{?}} ) \text{,} {freq}_{\text{?}} ( {phenotype}_{\text{?}} ) ) \} + \frac{1}{n} {\sum }_{j = 1}^{\text{?}} {max}_{1 \leq \text{?} \leq \text{?}} \{ Resnick ( {phenotype}_{\text{?}} , {phenotype}_{\text{?}} ) \times min ( {freq}_{\text{?}} ( {phenotype}_{\text{?}} ) , {freq}_{} ( {phenotype}_{\text{?}} ) ) \} ] ) + {w}_{2} \times ecdf ( D \text{:} \sqrt{{\sum }_{f} {{γ}_{f} ( {MRI}_{\text{?}}^{\text{?}} - {MRI}_{f}^{} )}^{2}} )$$
$$\text{?} \text{indicates text missing or illegible when filed}$$
(where ecdf(x; z) is is as an empirical cumulative distribution function for z, P is an input patient, D is a type of disease, Pr( ) is probability, w0 is a weight for genes and gene variants, w1 is a weight for phenotype information, w2 is a weight for MRI data, and w0+w1+w2=1 is satisfied, and each variable is defined as follows:
T: The number of prediction tools PathoPred that predict the pathogenicity of genetic variants,
θₜ: Weight for the t-th prediction tool PathoPredt Σθₜ=1
vᵢⱼ: j-th variant of patient P for the i-th gene reported to induce disease D
m: The number of phenotypes observed in patient P
n: The number of phenotypes reported in disease D
phenotype_{Pi}: i-th phenotype of patient P
phenotype_{Dj}: j-th phenotype reported in disease D
freq_{D}(phenotype): Frequency of phenotypes reported in disease D
MRI_{f}^{P}: f-th feature of patient P MRI data vector
MRI_{f}^{D}: f-th feature of disease D MRI data vector
γ_{f}: Weight for f-th feature of MRI data vector Σγ_{f}=1).
